(19) United States
(12) Patent Application Publication
(10) Pub. No.: US 2009/0130025 A1
(43) Pub. Date: May 21, 2009

(54) APPARATUS AND METHODS FOR THE PRODUCTION OF ULTRASOUND CONTRAST AGENTS

(75) Inventors: Marcel Rene Bohmer, Eindhoven (NL); Reinhold Wimberger-Friedl, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/577,820

(22) PCT Filed: Oct. 25, 2005
(86) PCT No.: PCT/IB05/53488

§ 371 Date: Apr. 24, 2007

(30) Foreign Application Priority Data
Oct. 29, 2004 (EP) 04105415.6

(51) Int. Cl. A61K 49/22

(52) U.S. Cl. 424/9.52

(57) ABSTRACT

Disclosed is a method of making gas bubbles in a liquid, the bubbles having substantially uniform size suitable for responding to ultrasound or other diagnostic tools. Gas (P2) is forced through one or more pores or nozzles, into the liquid (P1), the nozzles or pores being of substantially uniform diameter, the flow of the gas being controlled to thereby cause the formation of substantially monodisperse gas bubbles in the liquid. Moreover, an apparatus is disclosed for making a suspension of gas bubbles in a liquid of a size suitable for responding to ultrasound or other diagnostic tools. Said apparatus comprises means for forcing a gas through an array of nozzles or pores into the liquid, the nozzles or pores being of substantially uniform diameter, and first means for controlling; a flow parameter of the gas so that gas is suspended as substantially monodisperse gas bubbles in the liquid. Furthermore a kit for preparing a dispersion of gas bubbles of substantially uniform size suitable for ultrasound purposes, is disclosed.

The present application relates to ultrasound contrast agents and their preparation and to medical imaging using the ultrasound agents as well as an apparatus for generating contrast agents.

Commercially available ultrasound contrast agents are gas bubbles smaller than 10 micron in diameter. To increase their lifetime in the circulation they can be provided with a shell that can comprise proteins, especially human serum albumin, lipids and/or biodegradable polymers. To improve the lifetime in the circulation even more, gases are used that have a very low solubility in blood plasma, the mostly used gases are C₃F₈, C₄F₁₀ and SF₆. For targeted contrast agents liquid perfluorocarbons like perfluorooctanebromide have been used as well.

Due to the preparation conditions the size distributions of ultrasound contrast agents are normally rather wide, especially for lipid based agents. Where kits are used to generate the contrast agent on site, gas and liquid, are mixed by shaking. The liquid can contain precursors for the shell material. Either the complete kit can be sterilized or the kit and components can be sterilized separately before filling of the kit.

If kits as mentioned above are used for the preparation of contrast agent, the control of the mixing of the components and especially the gas into the liquid is poor. It is very difficult or not possible with currently available kits to generate a contrast agent with a narrow size distribution but there is a need to provide such a kit especially for targeted or drug-carrying contrast agents.

It is known from literature that forcing a liquid and a gas through an orifice together gives well-controlled bubble liquid suspensions, see Gordillo et. al, Physics of Fluids 16 (2004) p. 2828. Liquid and gas are included in the same compartment. The compositions used and the sizes reported are not directly suitable as an ultrasound contrast agent.

The object of the present invention is to provide improved ultrasound contrast agents and their method of preparation and to medical imaging or diagnosis using the ultrasound agents. Another advantage of the kits provided by the present invention is that they can be used on site in the form of a cartridge preferable in combination with a bench-top type of apparatus that supplies the pressure, pumps the liquid and may also supply the gas.

According to a first aspect, the invention provides a method of making gas bubbles in a liquid with a narrow size distribution, the bubbles having a size suitable for responding to ultrasound or other diagnostic tools, by forcing a gas through one or more pores or nozzles, into the liquid, the nozzles or pores being of substantially uniform diameter, the flow of the gas being controlled to thereby cause the formation of substantially monodisperse gas bubbles in the liquid. A flow parameter such as the pressure of the gas can be controlled. A flow rate of the liquid across the one or more nozzles or pores may be controlled so that shear forces at the one or more nozzles or pores assist the formation of the gas bubbles, e.g. effectively remove the bubbles from the orifices of the pores or nozzles. The flow of the liquid is advantageous as it exerts a force on the bubble being formed and controls the break-off when a certain size is reached.

The above method can enable a more monodisperse dispersion of gas bubbles to be created. This can be useful not only for ultrasound but other imaging techniques, and for drug delivery using ultrasound or other techniques.

An additional feature of the present invention is that the array of pores or nozzles comprises an etched array in a suitable substrate, e.g. in a semiconductor material such as silicon.

The nozzles or pores can be provided by membranes of controlled porosity, microchannels and SPG (Shirasu-porous glass) membranes.

Another such additional feature is the pores being orientated at an angle, not perpendicular to the flow of the liquid. This can be advantageous for the “snap-off” of partly formed droplets of the second liquid by the first liquid because the formed droplet will have a region with higher curvature.

Another such additional feature is the pores or nozzles having a coating to alter a wetting property.

Another additional feature is the dispersion comprising a contrast agent suitable for diagnostic imaging

Another additional feature is that the although no gas bubbles are formed before a pressure is applied, the gas is in contact with the liquid and therefore the liquid will be saturated with the gas.

Another aspect provides an apparatus for carrying out the method. In particular the present invention provides an apparatus making a suspension of gas bubbles in a liquid of a size suitable for responding to ultrasound or other diagnostic tools, comprising:

means for forcing a gas through an array of nozzles or pores into the liquid, the nozzles or pores being of substantially uniform diameter, and first means for controlling a flow parameter of the gas so that gas is suspended as substantially monodisperse gas bubbles in the liquid. To nucleate gas bubbles the Laplace pressure must be overcome, which is related to the surface tension and the pore diameter.

A second controlling means may be provided for controlling a flow rate of the liquid across or into the nozzles or pores so that shear forces at the nozzles or pores assist the gas to be suspended as substantially monodisperse gas bubbles in the liquid.

The present invention provides, in a further aspect, a cell for particle generation which can be used for example in a cartridge for the generation of a contrast agent in the form of capsules, the cell having a separator of a well-controlled porosity for separating a gas from a liquid. The kit can include a first source for the gas and a second source for the liquid. The liquid preferably contains the precursor for the shell material of the capsules. The separator can be any suitable microporous membrane such as a Shirasu Porous Glass (SPG) membrane or a microporous alumina membrane, or can comprise a etchable material such as, e.g. silicon, containing etched microchannels, or it can comprise a bundle of capillaries, e.g. hollow needles arranged in an array. The nozzles may project from the substrate in which they are held. Alternatively a porous polymer membrane such as a nucleopore filter can be used. The cell, containing at least a liquid and a gas in use, and a separator such as described above, is optionally further equipped with a means to develop a well-defined flow parallel to the membrane. By this flow the gas bubbles will be dislodged once they have reached a critical size, leading to gas bubbles with good uniformity. The presence of a shell forming material, phospho lipids, polymers and/or proteins in the liquid will stabilize the gas bubbles. The pore size and shape, the concentration of shell forming material present, the applied pressure and the liquid velocity parallel to the porous structure determine the particle size achieved.

The kit can be equipped with additional ports or compartments for additives, either as solids or as solutions. The kit can be equipped with a septum for injection of additional components to allow for a post-treatment, for instance a reaction to attach a ligand such as an antibody, antibody fragment or peptide to the contrast agent particles.

The kit can be supplied as a single use item, containing all the chemicals and means for bubble formation as a disposable cartridge or can be combined with a device for externally controlled application of a pressure on the gas and/or the liquid and a pump to circulate the liquid at the desired speed.

The present invention can provide contrast agents with improved physical and chemical properties, for instance an improved size distribution, well-defined shell properties, and an improved biodegradability as phagocytosis depends on size and surface properties. While the use as normal contrast agents is not that demanding, the present invention allows use in molecular imaging and drug release that require designated particles with a narrow size distribution, e.g. monodisperse and a well-defined shell elasticity.

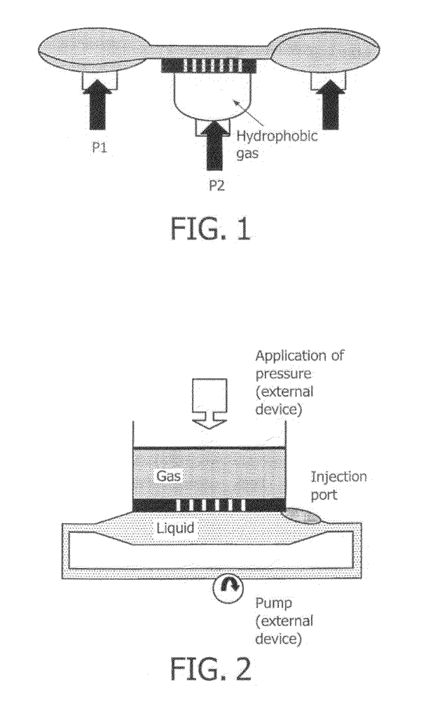

FIG. 1 shows a kit according to an embodiment of the present invention.

FIG. 2 shows a further kit in accordance with an embodiment of the present invention.

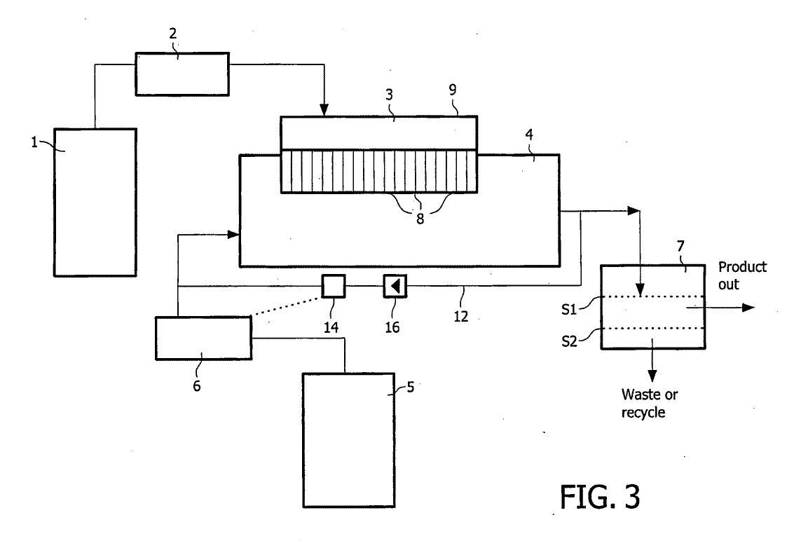

FIG. 3 is a general arrangement of an apparatus according to an embodiment of the present invention.

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

The term “comprising”, used in the claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

The present invention provides a method of making a dispersion of capsules in a liquid, the capsules having a size suitable for responding to ultrasound or other diagnostic tools, by forcing a gas through one or more nozzles or pores, e.g. through an array of nozzles or pores into the liquid. In embodiments of the present invention gas bubbles are formed directly, a type of particle sometimes referred to as gas filled liposomes, although liposomes generally encapsulate water and not oil or gas. The present invention includes the use and production of microbubbles or microballoons.

The nozzles or pores used to generate the gas filled bubbles are usually substantially uniform in diameter, the pressure of the gas and a flow rate of the liquid across the nozzles or pores is preferably arranged so that shear forces or convection at the nozzles or pore openings cause the gas to be suspended as substantially monodisperse bubbles in the liquid. These bubbles are then stabilized by the amphiphilic molecules present in the solution to avoid coagulation. One method of creating pores is with dry or wet etching. Very regular arrays of pores are created in a substrate, e.g. a rigid substrate such as a semiconductor substrate such as monocrystalline silicon or silicon-on-insulator wafers, or in any other suitable substrate, e.g. plastic, glass, quartz or a metal such as copper. The pores may also be made by any other suitable technique.

The pores preferably have diameters 5 micrometer or preferably smaller, a pitch of 10-20 micrometer and depths from 10 to over 25 micrometer in any suitable substrate, e.g. in monocrystalline silicon or silicon-on-insulator wafers or glass or metal substrates. Nucleopore membranes can be used, e.g. pore diameters of 200 nm are well suited for this.

The narrow pores serve as fine channels through which the gas can be pressed. The gas enters on the backside and leaves at the frontside, where it flows into the liquid. The liquid flows across the exit openings of the pores, i.e. flows parallel to the plane of the pore openings. There, due to the specific shear forces characteristic for the combination of liquids and gases, the exiting gas is suspended as highly monodisperse droplets. Such highly monodisperse droplets can be used directly or converted effectively as contrast agents for ultrasound imaging. The dimensions and shape of these pore arrays can be further tuned to tailor the particle size. Also the pore arrays may be fully or locally coated in order to change the wetting properties of the pores or pore outlets and their periphery in order to further tailor size and shape of the particles.

The highly monodisperse bubbles can be further processed to yield microbubbles with a shell, e.g. of a polymer or a phospholipid.

The gas bubbles formed have to be stabilized, during and after formation of the bubbles, adsorption of amphiphilic molecules, molecules having a hydrophilic and a hydrophobic part, has to occur to avoid coagulation. As the shell forming material may be present in the form of liposomes or vesicles this process is fairly slow. Therefore slow growth of the bubbles is preferred, which can be combined with the use of an elevated temperature to increase the kinetics of adsorption at the gas liquid interface. A suitable temperature would be 37° C., if created at this temperature the bubbles would not significantly expand upon injection.

The microchannels may be used to create small gas bubbles of, for instance, a perfluorocarbon gas and if such bubbles are led through a solution containing phospholipids, for instance, gas filled liposomes or microbubbles are generated and can be used as ultrasound contrast agent. This can help enable new options in early disease diagnostics by molecular imaging and targeted therapy.

The liquid contains the shell material, and if a drug-loaded agent is synthesized also the drug will be contained therein. The liquid is preferably an aqueous solution containing lipids such as phospholipids and cholesterol. These lipids will form liposomes or vesicles in the aqueous solution. By bubbling a gas through this solution, gas will be trapped in the liposomes or vesicles, creating the contrast agent. Hydrophilic or oil soluble drugs can be incorporated. In this case the liquid contains vesicles or liposomes that encapsulate a certain amount of oil, into which the hydrophobic drug is dissolved. Suitable drugs are anti-tumor drugs as paclitaxel and deoxyrubicin. Alternative shell material polymers may be used. For example, block-copolymers are very suitable. They form micelles or other self-associated structures of which the hydrophobic interior can be filled with gas. For the hydrophilic phase block poly-ethylene oxide is a preferred entity, as it is known to affect the biodistribution. Also in these association colloids oil soluble drugs can be incorporated. Finally polypeptides can be used that can be made partly hydrophobic, as an example partially denatured human serum albumin can be mentioned. It may be desirable to apply an additional force, on the liquid to quickly stabilize the bubbles with a layer of shell forming material.

A method to manufacture a regular injection pore array according to an embodiment of the present invention uses a substrate such as silicon into which an array of fine pores having a diameter typically a few micrometer with special shapes is formed, e.g. cylindrical, triangular, square, rectangular, hexagonal. These shapes can promote bubble detachment. An anisotropic etching technique can be used such as an RIE-etch to depths of several tens of micrometers in a conventional Si (100)-wafer. The bulk of the wafer etching is then done by wet-etching with KOH where the typical shape along the Si-(111) crystallographic planes automatically serves as a tapered inlet 50 for the gas. The resulting porous Si wafer or wafer part can be coated with special layers 40, e.g. oxide, nitride, etc. to further smoothen the fine pore walls. The wafer backside may be further mechanically strengthened by bonding the porous Si-wafer onto a support of a robust material with macro-openings corresponding to the tapered inlet openings of the Si-wafer. The orifices of the pores can protrude out of the substrate surface. In this way the droplet is created in an area of increased convection of the liquid while the shear stresses are still of the same order of magnitude as long as the protrusions are small compared to the total channel height of the liquid flow.

In one aspect the present invention provides a cell for particle generation for use in a kit for the generation of a contrast agent in the form of capsules, the cell having a separator of a well-controlled porosity for separating a gas compartment from a liquid compartment. The kit can include a first source for the gas and a second source for the liquid. All embodiments can include collection reservoirs, injection ports, temperature control.

The liquid preferably contains the precursor for the shell material of the capsules. The separator can be any suitable microporous membrane such as a Shirasu Porous Glass (SPG) membrane or a microporous alumina membrane, or can comprise a material such as a semiconductor, or any other etchable material e.g. silicon, containing microchannels, for instance, etched channels, or it can comprise a bundle of capillaries, e.g. hollow needles arranged in an array. Alternatively a porous polymer membrane such as a nucleopore filter can be used. The cell as shown schematically in FIG. 1, contains at least a liquid (1), a gas (2) in use, and is provided with a separator such as described above. It can be further equipped with a means to develop a flow parallel to the membrane. The flow is preferably well-defined. By this flow the gas bubbles adhering to the separator will be dislodged once they have reached a critical size, leading to gas bubbles with good uniformity. The presence of a shell forming material, such as phospholipids, polymers and or proteins in the liquid (1) can be used to stabilize the gas bubbles. The pore size and shape, the concentration of shell forming material present, the applied pressure and the liquid velocity parallel to the porous structure determine the particle size achieved.

The kit can be equipped with additional compartments for additives, either as solids or as solutions. The kit can also be equipped with a septum for injection of additional components to allow for a post-treatment, for instance a reaction to attach a ligand such as an antibody, antibody fragment or peptide.

The kit can be supplied as a single use item, or as a cartridge combined with a device for controlled application of a pressure on the gas and/or the liquid and a pump to circulate the liquid at the desired speed.

Ultrasound contrast agents are injected into a patient at concentrations of about 10⁸ to 10⁹ particles per injection. A desired injection volume is for instance 1 ml. Assuming 10⁹ particles of an ultrasound contrast agent with a particle diameter of 4 micron, this would imply that a total gas volume of 30 microliter has to inserted which would correspond to 3% in volume. A kit in accordance with the present invention is preferably constructed so that it can allow for volume changes of a few percent. This can be achieved by using flexible tubing, or deformable membranes, e.g. of cross-linked polymers preferably with a low glass transition temperature. Preferred polymers are polyolefins and polyurethanes.

Embodiments of the present invention make use of well-defined pores or nozzles through which a gas is passed in combination with a liquid flow of a liquid parallel to the separator surface. A controlled pressure of the gas provides emulsions with a narrow size distribution. The pores or nozzles in the separator preferably have a smaller effective diameter than the size of the gas bubble to be formed. Preferred diameters are smaller than 3 microns, more preferable smaller than 2 microns or smaller than 1 micron. Although a well-defined porosity is needed, the pores are not necessarily cylindrical. Shapes with rather pronounced edges or regions of high curvature can be used to regulate the droplet break-off process, as at this point the highest Laplace pressure will exist.

To better prevent the liquid from entering into the gas compartment the gas side of the separator is preferably hydrophobized, or alternatively a hydrophobic material is chosen for the separator, or alternatively the channels are designed to have a sharp transition in diameter which leads to contact line pinning of the liquid. To be effective the hydrophobization should include, for example, the outermost part of the pore or nozzle wall at the gas side of the separator. This hydrophobic layer also prevents the liquid from entering the gas compartment during transport and storage. Numerous materials can be used to make the surface of a material such as glass or silicon hydrophobic, for instance organosilanes can be deposited from a liquid or vapour phase. Fluorosilanes can be applied to increase the hydrophobicity. SF₆/C₄F₈ chemistry, which is also used in the preparation phase of the etching procedures, is also an excellent way to create a hydrophobic surface. Both liquid and vapour phase deposition techniques can allow for hydrophobization of complex geometries, so the pore walls can be hydrophobized. The liquid side of the separator is preferably hydrophilic. This side has to be shielded from the surface modification reaction, for instance using a removable foil during the reaction or partial immersion into a liquid during this reaction. The other parts in contact with liquid can be made hydrophilic as well, if hydrophobic parts are exposed there is a risk that formed gas bubbles will stick to these parts. Pegylated polymers or pegylated lipids are excellent materials to decrease the adhesion to a surface. These molecules will often be present in a formulation for ultrasound contrast agents to regulate the biodistribution and pre-treatment of the liquid compartment of the kit with a solution of these materials, or allowing a negligible fraction of the added phospholipid to be adsorbed on the walls is efficient to make the compartment surface hydrophilic.

The gas compartment can be pressurized externally, pressing the gas through the pores leads to a change in volume of the liquid, which will now also contain gas. The change in volume can be allowed for by using volume adaptive means such as a bellows. It is practical not too include all gas present in the reservoir in the liquid phase but to apply a defined pressure over a predetermined amount of time.

The kit can also have at least one injection port through which additional components can be added. The kit can also have a further port (or uses the injection port) through which the produced liquid with contrast agent can be extracted.

As an additional option the kit can include a reservoir for collection of the produced contrast agent. Such a collection can take place on the principle of existence of a density difference between contrast agent and suspending liquid. In a thin cell the contrast agent will quickly be present in the top layer. By opening a valve, the contrast agent can be collected.

### EMBODIMENT 1

In a first embodiment the kit can be constructed without external pump and only the provision of external pressure is needed. The embodiment is shown schematically in FIG. 1. A gas containing space or compartment is located at the bottom or underside of the kit. This is preferred because the microbubbles produced will float and because of that they will not interfere with bubbles that still adhere to the separator because they have not yet reached their critical size. The surface modification described above will make it possible to keep the gas at the bottom side. A wall, or part of the wall, of the gas compartment is formed by a gas-tight deformable membrane. These flexible membranes on the liquid and gas compartment, shown as curved surfaces, allow for exertion of an external pressure.

By exerting a pressure of gas on this membrane, and maintaining this pressure above a critical value determined by the nucleation of gas bubbles which is determined by the Laplace pressure, gas bubbles can be formed.

The liquid compartment has at least two parts equipped with a flexible membrane. These parts separated by a microchannel in which the membrane constitutes part of the channel wall. By applying a pressure difference between the two parts a well-defined flow can be established for the liquid. Preferably there is a constant gap between separator and the opposite wall. By applying a pressure on one of the membranes, liquid is forced to flow from one part of the liquid compartment on the one side of the separator to the other part of the compartment on the other side of the channel, passing the slit with the separator. To control the flow, either the pressure or the stroke is controlled. In passing the slit with the separator gas bubbles can be dislodged. If desired the liquid can be forced to pass the slit more than a single time by reversing the pressure gradient, e.g. by applying a pressure on the other side of the liquid compartment. These steps can be repeated until the contrast agent is ready for use. The kit can be used in combination with an apparatus that applies and measures the pressures required.

### EMBODIMENT 2

In a second embodiment the gas is not only present in a gas compartment, but gas can also be supplied externally, preferably incorporated in an apparatus that also controls the fluid flow. Compared to embodiment 1 this has the advantage that less stringent demands are placed on the permeability of the entire kit for gas. The disadvantage is the more complicated interface which has to be supplied by the instrument. The closed kit is placed in an apparatus, which has a designated volume that is purged with gas. Subsequently the kit is opened and by a controlled pressure gas is purged through the array of pores. Preferably the same apparatus takes care of the liquid flow along the separator.

### EMBODIMENT 3

In a third embodiment the liquid is not pressed from one side to the other but circulated using an external pumping device. In this embodiment polymer membranes are not necessary, for example flexible tubing is sufficient. As the total volume change is about 3%, the flexible tubing will allow for the change in volume. It is schematically shown in FIG. 2.

### EMBODIMENT 4

FIG. 3 is a schematic diagram of an apparatus for producing gas bubbles in accordance with another embodiment of the present invention. A source of gas is shown with reference number 1. The gas in the source 1 is fed by a pump (not shown) to a head 3, which comprises nozzles or pores 8 and is located in a container 9. The present invention includes that each nozzle or group of nozzles has a separate source of gas and each nozzle or group of nozzles is controlled separately. Alternatively, all of the pores or nozzles may be fed from a single source and controlled by a single controller. A flow parameter of the gas is controlled by a controller 2 which may be a pressure controller. The controller 2 may be a closed loop controller which receives an input from a pressure sensor (not shown) in the gas loop and controls the flow of gas, e.g. by controlling the pump or a valve to meter gas to the head 3 at the correct pressure/flow. The liquid is provided in a source 5 and is fed to another input of chamber 9 by means of gravity or via a pump (not shown). The feed of the liquid generates a flow of liquid across the front ends of the nozzles 8. The flow of the liquid is controlled by a controller 6. The controller 6 may be a closed loop controller which receives an input from a flow sensor (not shown) in the liquid loop and controls the flow of the liquid, e.g. by controlling the pump or a valve to meter liquid to the container 9 at the correct pressure/flow. The particles are collected in chamber 7. Further sizing of the particles may be performed, e.g. by an oversize sieve S1 which holds back oversized particles and/or an undersized sieve S2 which allows too small particles to be flushed from the system. Instead of the sieves S1 and S2 any other fractionation based on particle density may be used. Another method of fractionation is to make use of the fact that the flotation velocity depends on the particle size.

The nozzles 8 can be any of the nozzles described in embodiments of the present invention. The nozzles or pores are of substantially uniform diameter, and the controllers control a flow parameter of the gas and a flow rate of the liquid across the nozzles or pores so that shear forces at the nozzles or pores cause the gas to be suspended as substantially monodisperse gas bubbles in the liquid.

The flow of gas to the nozzles may be continuous or be determined by mechanical or electromechanical pulses. The pulses do not need to be sufficient to generate free floating bubbles. Due to the flow of liquid past the opening of the nozzles, gas which has formed a convex meniscus by a smaller pulse can be dragged away by the flow of the liquid at a time when the meniscus has not reached sufficient size for the bubble to break free if the flow of liquid were not present. The present invention also includes the controlling the gas in a continuous flow to generate bubbles. In this case, due to the flow of liquid passed the opening of the nozzles, gas which has formed a convex meniscus by constant flow can be dragged away by the flow of the liquid at a time when the meniscus has not reached sufficient size for the droplet to break free if the flow of liquid were not present.

In the collection compartment separation of contrast agent and liquid can be carried out based on gravity. The pure liquid at the bottom of this compartment can be recirculated towards the entrance compartment and pumped through the channel again. In this way all the liquid will be effectively loaded with contrast agent.

The apparatus of FIG. 3 may be modified a way to allow the liquid to pass the porous surface more than once: therefore it can collect more gas bubbles because the number of passes of liquid on the membrane can be varied independently. For example, a bypass 12 can be provided which allows the continuous phase, i.e. the liquid to pass the porous surface more than once. The flow may be controlled by a one way flow device 16 and by a valve 14 which may be controlled by the controller 6 or may be controlled separately.

Applications of the microbubbles according to the present invention include ultrasound contrast agents, especially targeted ultrasound contrast agents. Various ultrasound applications can benefit from the better acoustic properties of contrast agents with a well-defined size distribution and consistent shell properties in accordance with the present invention. Monodisperse ultrasound contrast agents have many advantages. As harmonic peaks are more distinct compared to polydisperse agents, the contrast to tissue ratio improves. This advantage can be exploited further if a mixture of two monodisperse contrast agents with a distinctly different size is used: the presence of two harmonic peaks proves that one is looking at the contrast agent. The performance of pressure measurements using ultrasound contrast would become possible: The resonance frequency of a bubble is to a good approximation given by the Minnaert frequency. The resonance frequency in rad/s is given by:

$${ω}_{0} = \frac{1}{R} \sqrt{\frac{3 p}{ρ}} \qquad ( 1 )$$

where p is the pressure, R the radius of the bubble and ρ the density of the fluid. For an adiabatic case the 3 in the numerator has to be replaced by 3γ, with γ being the polytropic gas coefficient (e.g. 1.4 for air). For an air bubble with radius of 2 μm, in water, under atmospheric conditions, ω₀=8.7·10⁶ rad/s, which is 1.37 MHz.

Again using a mixture of two distinct sizes would improve the quality of the pressure measurement.

For targeted contrast agents a tight size distribution can lead to a discrimination between adhered and non-adhered contrast agent. For a contrast agent with a wide size distribution this has been shown by Dayton et al. Molecular Imaging vol 3 no 2 Apr. 2004, p 125-134. They study the accumulation of contrast agent targeted to αᵥβ₃ integrins and observe a shift in the echo spectra to lower frequencies for adhering contrast agent. A shift in the direction observed by Dayton et al is predicted by Scott in J. F. Scott “Singular perturbation theory applied to the collective oscillation of gas bubbles in a liquid”, J. Fluid Mech. 113, 487-511 (1981). This theory is based on potential flow calculations but can reasonably well extrapolated to smaller bubbles as well. A function is disclosed that describes the decrease of the resonance frequency close to a wall, or for the similar case of two equally sized bubbles. When the bubble touches the wall a resonance frequency of 0.83ω₀ was determined. For high surface coverage an additional decrease can be expected, Duineveld, J. Acoust. Soc. Am. 99, 622-624, 1996, demonstrated the effect of a decrease of the resonance frequency of two equally sized bubbles experimentally. If monodisperse targeted contrast agents are used, the distinction between bound and unbound contrast agent is expected to become more evident compared to the result by Dayton et al. The use of monodisperse contrast agents allows the shift to be studied more quantitatively and potentially extract clinically relevant information. Also in this case a mixture of distinctively different sizes could be employed targeted to different markers, for instance VEGF and αᵥβ₃ integrins.

For drug delivery a better control of the size distribution using the proposed preparation methods has the advantage that the amount of drug incorporated is also well controlled. Drug release can therefore be quantified. With uniform shell properties of the agents, release by cavitation is also under better control than with a polydisperse sample.

### Concluding Remarks

Well-defined ultrasound contrast agents made in the way described above can make it possible to obtain superior images, even for very small blood vessels. Furthermore, applications in ultrasound imaging, especially targeted ultrasound imaging as well as in therapy, especially targeted and localized therapy are provided by the present invention. Both applications rely on the availability of well-defined gas bubbles. As an example of targeted ultrasound imaging, the present invention can be used in any specific pathology like vulnerable arterial plaque, which plays a major role in acute cardiovascular disease, in blood vessels. Ultrasound-assisted local drug delivery is a second and very important application, which is enabled by the proposed particle manufacturing methods. In accordance with this embodiment micro-bubbles made in accordance with the present invention are loaded with drugs.

1. A method of making gas bubbles in a liquid, the bubbles having substantially uniform size suitable for responding to ultrasound or other diagnostic tools, by forcing a gas through one or more pores or nozzles, into the liquid, the nozzles or pores being of substantially uniform diameter, the flow of the gas being controlled to thereby cause the formation of substantially monodisperse gas bubbles in the liquid.

2. The method according to claim 1 where the liquid is flowed past the nozzles or pores and the shear flow of the liquid is controlled to assist the formation of the substantially monodisperse gas bubbles in the liquid.

3. The method according to claim 1 wherein the pores or nozzles in an alumina, silicon, Shiraus Porous Glass or polymeric membranes.

4. The method according to claim 1 wherein the pore or nozzle diameters are smaller than 5 micrometer

5. The method according to claim 3 further comprising a hydrophobic surface on the gas input side of the porous membrane

6. The method according to claim 1 the liquid comprising amphiphilic molecules

7. The method of claim 6 the amphilic molecules being lipids and or biodegradable block-copolymers.

8. An apparatus making a suspension of gas bubbles in a liquid of a size suitable for responding to ultrasound or other diagnostic tools, comprising:
means for forcing a gas through an array of nozzles or pores into the liquid, the nozzles or pores being of substantially uniform diameter, and first means for controlling a flow parameter of the gas so that gas is suspended as substantially monodisperse gas bubbles in the liquid.

9. A kit comprising compartments for a gas and a liquid, separated by a material of controlled porosity, through which the gas can be pressed into the liquid to yield a dispersion of gas bubbles of substantially uniform size suitable for ultrasound purposes.

10. The kit as in claim 9 that can be subjected to controlled flow of gas by the application of an external pressure

11. The kit as in claim 9 that can be subjected to controlled flow of liquid by an external pressure or a pump.

12. The kit as in claim 9 having flexible polymeric membranes (for the exertion of pressure)

13. The kit as claimed in claim 9 having flexible tubing to allow for volume changes by pressing the gas into the liquid.

14. The kit as claimed in claim 9 having an injection port to add additional components or extract the dispersion of gas bubbles.

15. The kit as claimed in claim 9 having a collection reservoir.

16. The kit with a collection reservoir as claimed in claim 9 oriented such that separation takes place on density.

17. A kit as claimed in claim 9 the kit being a cartridge that can be inserted in a table top type of apparatus that supplies external pressure, pumps the liquid and may supply the gas.
